(19) United States
(12) Patent Application Publication
(10) Pub. No.: US 2012/0302831 A1
(43) Pub. Date: Nov. 29, 2012

(54) GUIDE APPARATUS FOR ENDOSCOPE

(76) Inventors: Tsuyoshi Ashida, Kanagawa (JP); Takayuki Nakamura, Kanagawa (JP); Shinichi Yamakawa, Kanagawa (JP); Yasunori Ohta, Kanagawa (JP)

(21) Appl. No.: 13/479,812

(22) Filed: May 24, 2012

(30) Foreign Application Priority Data
May 25, 2011 (JP) 2011-117067

(51) Int. Cl. A61B 1/00

(52) U.S. Cl. 600/114

(57) ABSTRACT

An endoscope has a head assembly for entry in a body cavity. A guide apparatus for propulsion of the head assembly is provided, and includes a shaft sleeve, a support sleeve, a drive sleeve, drive gears, an endless track device, two intermediate gears, and a motor. Two wire devices are disposed to extend in an axial direction, have distal ends to which the intermediate gears are respectively secured, for transmitting rotation of the motor to the intermediate gears. In fail-safe control, if one of the wire devices is broken, the broken wire device is detected. When the broken wire device is detected, a limited mode is set for driving the endless track device with a normal one of the wire devices in limited performance relative to normal operation. In a preferred embodiment, the guide apparatus has a differential device and two brake devices.

### BACKGROUND OF THE INVENTION

1. Field of the Invention

The present invention relates to a guide apparatus for an endoscope. More particularly, the present invention relates to a guide apparatus for an endoscope, capable of fail-safe control in case of occurrence of failure in a wire device for driving.

2. Description Related to the Prior Art

An endoscope is widely used for medical diagnosis. An elongated tube or guide tube of the endoscope is entered in a body cavity of a patient. A CCD image sensor or other imaging device is incorporated in the elongated tube. An image of an object in the body cavity is created by the endoscope, and is display on a display panel. A doctor or operator observes the image of the object.

U.S. Pat. Pub. No. 2005/0272976 (corresponding to JP-A 2005-253892) discloses a propulsion assembly for propulsion of the endoscope in the body cavity. The propulsion assembly includes a sleeve and a plurality of endless belts or endless track device. The sleeve is mounted on the elongated tube of the endoscope. The endless belts are supported on the sleeve movably in an axial direction. An upper run (active run) of the endless belts is kept in contact with tissue of a gastrointestinal tract, and is moved endlessly. Thus, a tip of the endoscope is propelled according to friction between the endless belts and the tissue. It is possible for a doctor or operator with insufficient skill of manipulation to enter the endoscope of the gastrointestinal tract into the body cavity, even a sigmoid colon in a large intestine or the like having a tortuous form.

In the U.S. Pat. Pub. No. 2005/0272976, a motor rotates a wire device. A magnet fixed at a distal end of the wire device is rotated to move the endless track device endlessly by use of a idler roller formed from magnetic material. As there is load applied to the wire device constantly during the movement, breakage is likely to occur in the wire device. The endless track device cannot be turned around. It is extremely difficult to remove the propulsion assembly from out of the body cavity.

### SUMMARY OF THE INVENTION

In view of the foregoing problems, an object of the present invention is to provide a guide apparatus for an endoscope, capable of fail-safe control in case of occurrence of failure in a wire device for driving.

In order to achieve the above and other objects and advantages of this invention, a guide apparatus for an endoscope having a section of an elongated tube for entry in a body cavity is provided, and includes a propulsion assembly, having an endless track device, for mounting on a head assembly of the elongated tube, the endless track device moving endlessly in the body cavity to propel the head assembly. A motor is disposed discretely from the endoscope. Plural wire devices transmit rotation of the motor to move the endless track device. A differential device is disposed between the wire devices and the motor, for changing a rotational speed of the wire devices according to load applied to the wire devices. Plural brake devices brake respectively the wire devices. A failure detector, if one of the wire devices is broken, detects the broken wire device according to a change in the rotational speed. A controller actuates one of the brake devices corresponding to the broken wire device, to transmit rotation of the motor to a normal one of the wire devices.

The differential device is a differential gear device.

When the failure detector detects the broken wire device, the controller disables a normal mode for the motor without limitation, and sets a limited mode for limited operation of the motor relative to the normal mode.

In the limited mode, the motor is rotated backwards to move the elongated tube in a proximal direction.

In another preferred embodiment, in the limited mode, the motor is rotated at a low speed.

The failure detector includes plural speed sensors for detecting the rotational speed of respectively the wire devices. An arithmetic unit compares the rotational speed from the speed sensors, and determines that one of the wire devices of which the rotational speed is higher is the broken wire device if a difference in the rotational speed becomes equal to or more than a reference value.

The propulsion assembly includes a shaft sleeve for mounting on the head assembly of the elongated tube. A support sleeve is disposed around the shaft sleeve, for supporting the endless track device movably along inner and outer surfaces thereof. A driving device drives the endless track device.

The driving device includes a drive sleeve, surrounded in the support sleeve, supported around the shaft sleeve in a rotatable manner, and rotated by the wire devices. Worm gear teeth are formed around the drive sleeve. A barrel sleeve is disposed between the drive sleeve and the support sleeve. Plural drive gears are arranged on the support sleeve in a circumferential direction, rotated by the worm gear teeth, for endlessly moving the endless track device. Plural idler rollers are arranged on the support sleeve, for tensioning the endless track device in cooperation with the drive gears.

The barrel sleeve has three walls arranged triangularly as viewed in a cross section, and each one of the drive gears is disposed on one of the walls. The inner surface of the support sleeve is shaped triangularly to correspond to the barrel sleeve, and the outer surface is cylindrical.

The endless track device is a device having an annular surface and extending to cover the support sleeve, or includes plural endless belts.

In still another preferred embodiment, the controller increases a rotational speed of the normal wire device with the differential device.

In another preferred embodiment, furthermore, a motor driver drives the motor, and lowers a maximum of a current supplied to the motor when the limited mode is set.

Also, a safety control method for a propulsion assembly of an endoscope having a head assembly for entry in a body cavity is provided. The propulsion assembly includes a shaft sleeve for mounting on the head assembly, a support sleeve disposed around the shaft sleeve, an endless track device, disposed to extend along inner and outer surfaces of the support sleeve, for endlessly moving in an axial direction of the head assembly in contact with an inner wall of the body cavity, for propulsion of the head assembly, a driving device, disposed around the shaft sleeve, for driving the endless track device, plural intermediate gears for actuating the driving device by engagement therewith, a motor disposed outside the body cavity, and plural wire devices, disposed to extend in the axial direction, having distal ends to which the intermediate gears are respectively secured, for transmitting rotation of the motor to the intermediate gears. In the safety control method, if at least one of the wire devices is broken, the broken wire device is detected. When the broken wire device is detected, a limited mode is set for driving the endless track device with a normal one of the wire devices in limited performance relative to normal operation.

Also, a safety control computer executable program for a propulsion assembly of an endoscope having a head assembly for entry in a body cavity is provided. The propulsion assembly includes a shaft sleeve for mounting on the head assembly, a support sleeve disposed around the shaft sleeve, an endless track device, disposed to extend along inner and outer surfaces of the support sleeve, for endlessly moving in an axial direction of the head assembly in contact with an inner wall of the body cavity, for propulsion of the head assembly, a driving device, disposed around the shaft sleeve, for driving the endless track device, plural intermediate gears for actuating the driving device by engagement therewith, a motor disposed outside the body cavity, and plural wire devices, disposed to extend in the axial direction, having distal ends to which the intermediate gears are respectively secured, for transmitting rotation of the motor to the intermediate gears. The safety control computer executable program includes a detecting program code for, if at least one of the wire devices is broken, detecting the broken wire device. A setting program code is for, when the broken wire device is detected, setting a limited mode for driving the endless track device with a normal one of the wire devices in limited performance relative to normal operation.

Also, a safety control user interface for a propulsion assembly of an endoscope having a head assembly for entry in a body cavity is provided. The propulsion assembly includes a shaft sleeve for mounting on the head assembly, a support sleeve disposed around the shaft sleeve, an endless track device, disposed to extend along inner and outer surfaces of the support sleeve, for endlessly moving in an axial direction of the head assembly in contact with an inner wall of the body cavity, for propulsion of the head assembly, a driving device, disposed around the shaft sleeve, for driving the endless track device, plural intermediate gears for actuating the driving device by engagement therewith, a motor disposed outside the body cavity, and plural wire devices, disposed to extend in the axial direction, having distal ends to which the intermediate gears are respectively secured, for transmitting rotation of the motor to the intermediate gears. The safety control user interface includes a detecting region for, if at least one of the wire devices is broken, detecting the broken wire device. A setting region is for, when the broken wire device is detected, setting a limited mode for driving the endless track device with a normal one of the wire devices in limited performance relative to normal operation.

Consequently, fail-safe control in the propulsion with the guide apparatus is possible in case of occurrence of failure in a wire device for driving, because a remaining wire device different from the broken wire device can operate reliably in a safety condition.

### BRIEF DESCRIPTION OF THE DRAWINGS

The above objects and advantages of the present invention will become more apparent from the following detailed description when read in connection with the accompanying drawings, in which:

FIG. 1 is a side elevation illustrating an endoscope with a guide apparatus;

FIG. 2 is a block diagram illustrating circuit elements in the guide apparatus;

FIG. 3 is a perspective view illustrating an endless track device with the endoscope;

FIG. 4 is an exploded perspective view illustrating sleeves in the guide apparatus for the endless track device;

FIG. 5 is a vertical section illustrating the guide apparatus;

FIG. 6 is a cross section illustrating the guide apparatus;

FIG. 7 is a cross section taken on a line passing centers of the drive gears, illustrating the guide apparatus;

FIGS. 8A and 8B are a flow chart illustrating operation of the guide apparatus;

FIGS. 9A and 9B are a flow chart illustrating another preferred embodiment in which a slow mode can be set in a guide apparatus;

FIGS. 10A and 10B are a flow chart illustrating another preferred embodiment in which a low current mode can be set in a guide apparatus.

### DETAILED DESCRIPTION OF THE PREFERRED EMBODIMENT(S) OF THE PRESENT INVENTION

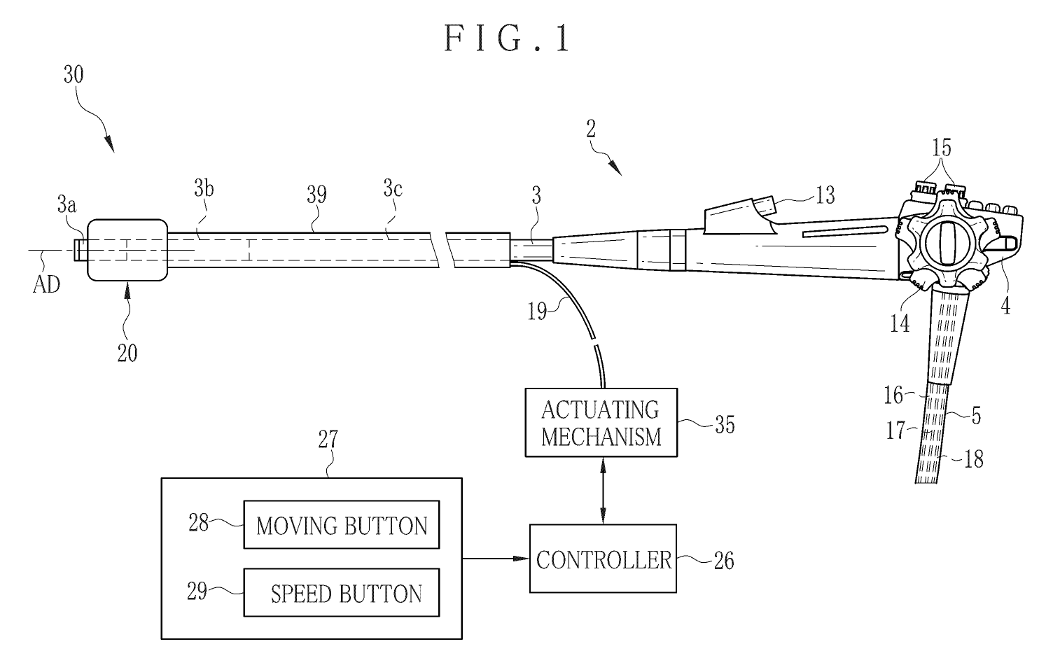

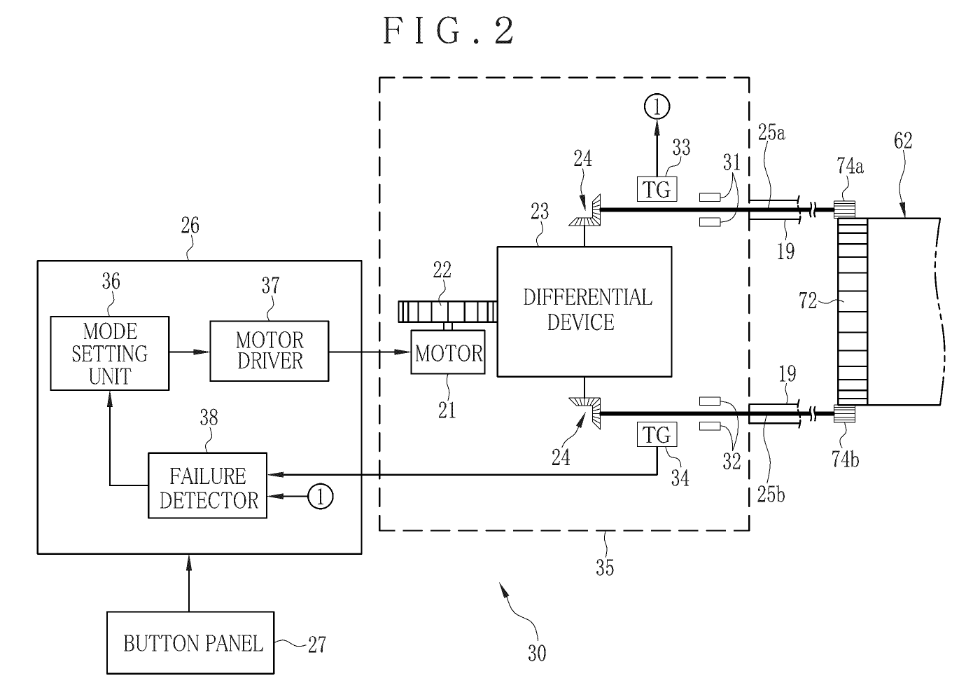

In FIGS. 1 and 2, an endoscope 2 includes a section of an elongated tube 3 or guide tube, a handle 4 and a universal cable 5. The elongated tube 3 is entered in a body cavity for imaging, for example gastrointestinal tract of a patient's body. The handle 4 is held manually by a doctor or operator and used for operating the elongated tube 3. The universal cable 5 connects the endoscope 2 to a processing apparatus, light source apparatus and fluid supply apparatus (all not shown).

The elongated tube 3 includes a head assembly 3a, a steering device 3b and a flexible device 3c. The head assembly 3a is a rigid device in which a CCD image sensor is incorporated. The steering device 3b is disposed at a proximal end of the head assembly 3a, and bendable up and down and to the right and left. The flexible device 3c extends from a proximal end of the steering device 3b with flexibility.

An imaging window 7, lighting windows 8a and 8b, and a distal instrument opening 9 are formed in the head assembly 3a of the elongated tube 3. The distal instrument opening 9 is used for protrusion of a tip of a forceps as a medical instrument. A nozzle spout of an end nozzle 10 is disposed on the head assembly 3a for ejection of air or water toward the imaging window 7.

The imaging window 7 is disposed on a distal side of the CCD image sensor for passing object light. The lighting windows 8a and 8b are arranged symmetrically with respect to the imaging window 7, and apply light from the light source apparatus to an object of interest in the body cavity.

An instrument channel extends from the distal instrument opening 9 in the proximal direction. A proximal instrument opening 13 is a proximal end of the instrument channel, and disposed in the handle 4. A medical instrument is entered through the proximal instrument opening 13. Examples of tips of such medical instruments are a forceps, injection needle, electrocautery device and the like.

The handle 4 includes steering wheels 14 and fluid supply buttons 15. The steering wheels 14 are rotatable for steering the steering device 3b up and down and to the right and left. The fluid supply buttons 15 are depressed for supplying air and water.

The universal cable 5 is connected with the handle 4. A fluid supply channel 16, a signal line 17, and a light guide device 18 are disposed through the universal cable 5. A proximal end of the fluid supply channel 16 is connected with the fluid supply apparatus. A distal end of the fluid supply channel 16 is connected to the end nozzle 10, so that the fluid supply channel 16 draws fluid from the fluid supply apparatus to the end nozzle 10. A proximal end of the signal line 17 is connected with the processing apparatus. A distal end of the signal line 17 is connected to a circuit for the CCD image sensor. A distal end of the light guide device 18 is connected with the lighting windows 8a and 8b. A proximal end of the light guide device 18 is connected to the light source apparatus, so that the light guide device 18 guides light to the lighting windows 8a and 8b.

A propulsion assembly 20 is a functional unit in a guide apparatus 30, and mounted on the head assembly 3a of the elongated tube 3 removably for propulsion (and return) of the elongated tube 3 in a gastrointestinal tract. An actuating mechanism 35 has a motor 21, which drives the propulsion assembly 20. A motor gear 22 is coupled with an output shaft of the motor 21. A differential device 23 or differential gear device is engaged with the motor gear 22. A transmission gear train 24 includes plural gears and is connected with the differential device 23. A first wire device 25a and a second wire device 25b for torque are connected to the differential device 23 by the transmission gear train 24, and transmit torque for propulsion of the propulsion assembly 20.

The differential device 23 is a differential gear set well-known in the mechanical field, and includes a link gear, a frame, a pinion gear, and first and second wire gears (all not shown). The link gear is meshed with the motor gear 22. The frame is attached to the link gear. The pinion gear is supported on the frame in a rotatable manner. The first and second wire gears are meshed with the pinion gear, and are coupled with respectively the wire devices 25a and 25b. The differential device 23 absorbs a rotational difference between the wire devices 25a and 25b. If an equal load is applied to the wire devices 25a and 25b, the first and second wire gears are rotated at an equal speed without rotating the pinion gear. If load applied to the second wire device 25b is different to that applied to the first wire device 25a, the pinion gear rotates according to the load to increase a rotational speed of one of the first and second wire gears in connection with the wire device 25a or 25b with a smaller load.

A protection sheath 19 covers the entirety of each of the wire devices 25a and 25b at least between the actuating mechanism 35 and the propulsion assembly 20. When the motor 21 is driven, the wire devices 25a and 25b rotate inside the protection sheath 19.

A controller 26 controls the motor 21. A button panel 27 is connected to the controller 26, and includes a moving button 28 and a speed button 29. The moving button 28 generates a control signal for advance, return and stop of the propulsion assembly 20. The speed button 29 generates a control signal for setting a movement speed of the propulsion assembly 20.

An overtube 39 covers an outer surface of the elongated tube 3. The protection sheath 19 is disposed between the overtube 39 and the elongated tube 3.

The actuating mechanism 35 includes a first brake device 31 and a second brake device 32. The first brake device 31 brakes the first wire device 25a. The second brake device 32 brakes the second wire device 25b. The brake devices 31 and 32 are controlled by the controller 26. A first tachogenerator (TG) 33 as a speed sensor detects a rotational speed of the first wire device 25a. A second tachogenerator (TG) 34 as a speed sensor detects a rotational speed of the second wire device 25b. The tachogenerators 33 and 34 generate direct current voltages in proportion to respectively the rotational speeds, and supply the controller 26 with the voltages.

The controller 26 includes a mode setting unit 36, a motor driver 37 for current generation, and a failure detector 38 or breakage detector for fail-safe control. The failure detector 38 detects breakage of the wire devices 25a and 25b. The failure detector 38 is an arithmetic unit including a subtractor and a comparator. The subtractor calculates a difference between the DC voltages of the tachogenerators 33 and 34. The comparator checks whether the difference is equal to or more than a threshold as a reference value by comparison. If both of the wire devices 25a and 25b are normal, their rotational speeds are equal because equal load is applied to those. If breakage of one of the wire devices occurs, the load to the broken wire device decreases extremely. The differential device 23 causes a normal one of the wire devices to stop or rotate slowly. The broken wire device rotates at a high rotational speed, so that there occurs a difference in the rotational speed between the wire devices 25a and 25b. When the difference in the DC voltage between the tachogenerators 33 and 34 becomes equal to or higher than the threshold, then the failure detector 38 determines that one of the wire devices at which the DC voltage is higher has broken. The failure detector 38 sends the mode setting unit 36 information of occurrence of the breakage and the particular broken wire device.

In response, the mode setting unit 36 disables the normal mode and sets a limited mode. In the normal mode, the motor 21 is rotatable in any one of the directions and any speed both according to input signals of the button panel 27. In the limited mode, the motor 21 is rotated in a limited manner by use of the motor driver 37 in consideration of safety in the body. The motor driver 37 is controlled in the limited mode to drive the motor 21 in a backward direction to remove the elongated tube 3 from the body cavity rapidly. Also, the mode setting unit 36 in the limited mode actuates one of the brake devices 31 and 32 for a broken one of the wire devices shortly before starting the backward rotation of the motor 21. The brake device 31 or 32 prevents the broken wire device from rotating idly. Rotation of the motor 21 is transmitted by a normal one of the wire devices to the propulsion assembly 20. Note that the brake devices can be actuated after rotating the motor 21 backwards. Also, the failure detector 38 may actuate the brake devices shortly setting the limited mode.

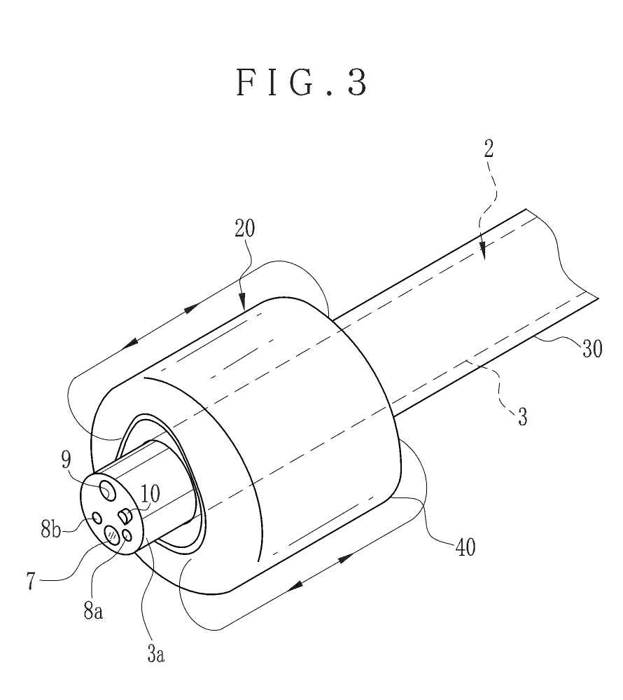

In FIG. 3, an endless track device 40 is incorporated in the propulsion assembly 20. An upper run (active run) 46 of the endless track device 40 contacts an inner wall of a gastrointestinal tract, and moves in a proximal direction for exerting force of propulsion for the elongated tube 3 of the endoscope 2. A support sleeve 42 is an outer sleeve for supporting the endless track device 40 movably in an axial direction AD by turn around. The endless track device 40 extends entirely for the length of the support sleeve 42. The arrows in FIG. 3 indicate directions of the turn around. The endless track device 40 is formed from flexible material. Examples of the material include polyvinyl chloride, polyamide resin, fluorocarbon resin, polyurethane resin, and other biocompatible plastic compounds.

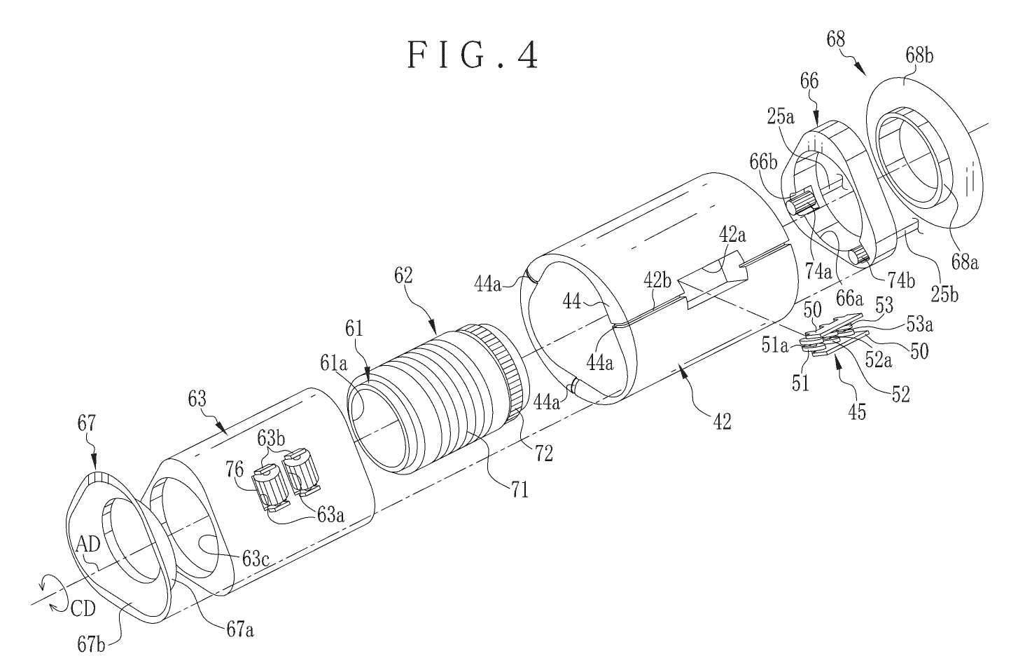

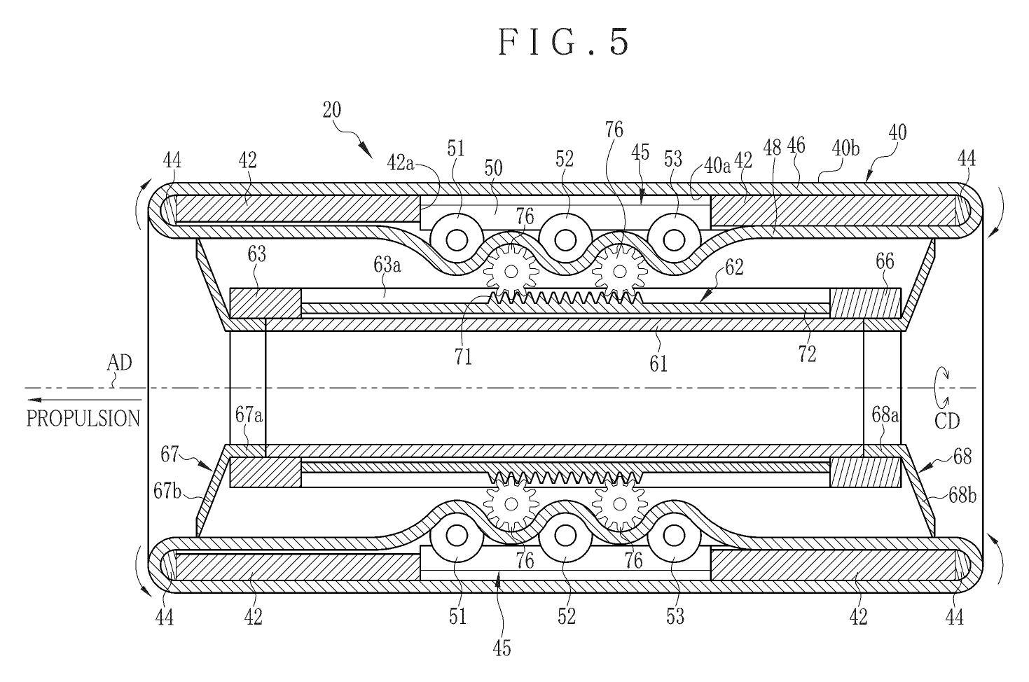

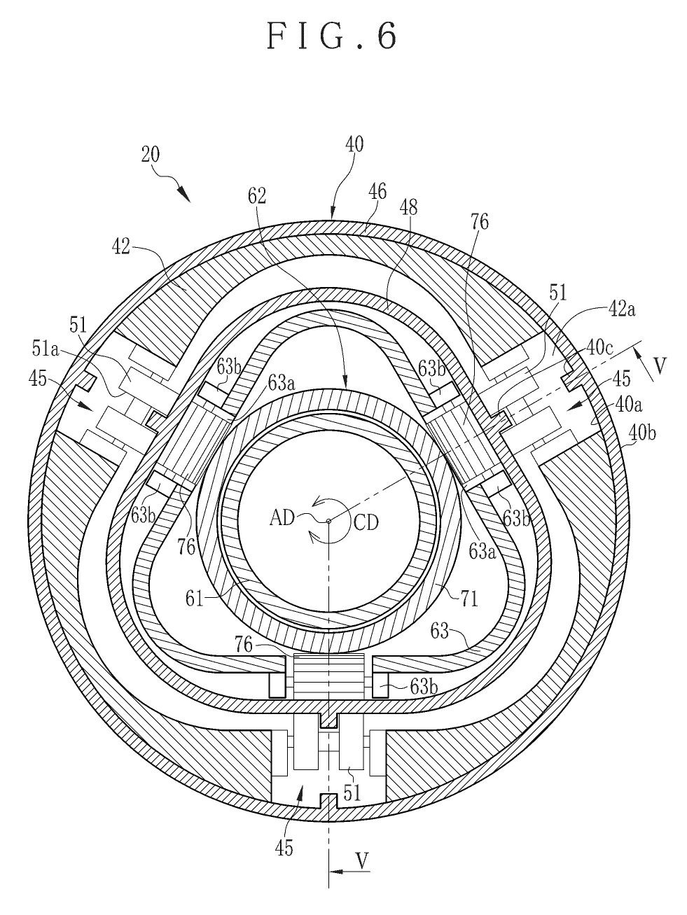

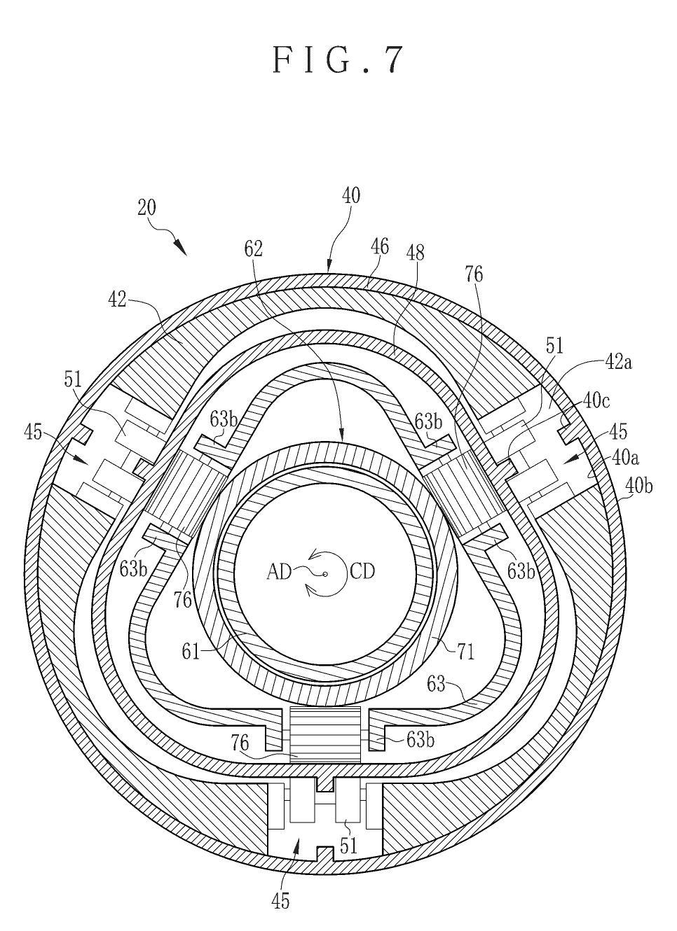

In FIGS. 4, 5, 6 and 7, an outer surface of the support sleeve 42 is circular as viewed in a cross section. An inner surface of the support sleeve 42 is triangular with curved corners as viewed in the cross section. Examples of a triangle of the triangular shape are an equilateral triangle and an isosceles triangle. Note that FIG. 5 is a vertical section taken on line V-V of FIG. 6.

For production of the endless track device 40, an original tube material with ends is prepared. The tube material is bent inside out to follow the shape of the support sleeve 42, and is wound about the support sleeve 42. Finally, the ends are attached to one another by thermal welding or the like, to obtain the endless track device 40 in an endless form.

A curved support ring with a slip characteristic is fitted on each of proximal and distal ends of the support sleeve 42, and includes a curved support surface 44 of an annular shape, which contacts an inner surface of a bend of the endless track device 40. The curved support ring is formed from a material with the slip characteristic for keeping the endless track device 40 movable smoothly. Examples of the material include nylon, polyetheretherketone (PEEK), tetrafluoroethylene polymer or Teflon (trade name), and the like.

A side opening 42a is formed in the support sleeve 42 at each of three portions of which an inner surface is flat. A roller unit 45 is attached to the inside of the side opening 42a for contacting the endless track device 40 in a movable manner. The roller unit 45 includes two support plates 50, a first idler roller 51, a second idler roller 52, and a third idler roller 53 or support rollers. The idler rollers 51-53 are arranged in the axial direction AD, and supported by the support plates 50. It is possible to secure the idler rollers 51-53 directly to the support sleeve 42 in a rotatable manner. The number of the positions of disposing the roller unit 45, although three according to the embodiment, can be determined two, or four or more for the purpose.

The endless track device 40 includes a lower run (return run) 48. An inner surface 40a of the lower run 48 contacts the idler rollers 51-53. Three portions of the endless track device 40 contacting the idler rollers 51-53 have a larger thickness than a remaining portion of the endless track device 40, and a higher rigidity than the same.

Roller grooves 51a, 52a and 53a are formed in respectively the idler rollers 51-53. Three engagement projections 40c or ridges are formed on the inner surface 40a of the endless track device 40. The engagement projections 40c extend fully with the length of the inner surface 40a. The roller grooves 51a, 52a and 53a receive respectively the engagement projections 40c in a slidable manner, and prevent the endless track device 40 from incidentally shifting in the circumferential direction CD. Also, a groove 42b is formed in the support sleeve 42. An end groove 44a is formed in the curved support surface 44. The grooves 42b and 44a receive each of the engagement projections 40c. Note that lubricant agent as a coating is applied to surfaces of the grooves 42b, 44a, 51a, 52a and 53a and the engagement projections 40c for a high slip characteristic.

The support sleeve 42 contains a shaft sleeve 61, a drive sleeve 62 and a barrel sleeve 63. The shaft sleeve 61 is mounted on the head assembly 3a of the endoscope 2 by receiving the same inside. The drive sleeve 62 is supported around the shaft sleeve 61 in a rotatable manner. The barrel sleeve 63 contains the shaft sleeve 61 and the drive sleeve 62.

An end ring 66 is secured to a proximal end of the barrel sleeve 63. A distal cover flange 67 is secured to a distal end of the barrel sleeve 63, and prevents entry of body tissue of the gastrointestinal tract. A proximal cover flange 68 is secured to a proximal end of the barrel sleeve 63 for the same purpose.

The drive sleeve 62 is supported around the shaft sleeve 61, and rotates around the axial direction AD. The drive sleeve 62 includes worm gear teeth 71 and spur gear teeth 72. The worm gear teeth 71 are a thread defined around the axial direction AD. The spur gear teeth 72 are arranged in a circumferential direction CD. The spur gear teeth 72 are formed at a proximal end of the drive sleeve 62. A first intermediate gear 74a of the first wire device 25a is meshed with the spur gear teeth 72. A second intermediate gear 74b of the second wire device 25b is meshed with the spur gear teeth 72. The first and second intermediate gears 74a and 74b are rotated by respectively the wire devices 25a and 25b, and cause the spur gear teeth 72 to rotate, for the drive sleeve 62 to rotate. Holes (not shown) are formed through the proximal cover flange 68, and cause the wire devices 25a and 25b respectively to pass.

The barrel sleeve 63 is in a shape of a triangular prism with curved corners. Side openings 63a are formed in flat walls of the barrel sleeve 63. For the driving device, drive gears 76 or engagement rollers (wheels) are disposed in respectively the side openings 63a. A holder bracket 63b is formed on the barrel sleeve 63, and supports each of the drive gears 76 in a rotatable manner. Each of the drive gears 76 is disposed between the idler rollers 51 and 52 or between the idler rollers 52 and 53.

An outer surface 40b of the endless track device 40 is contacted by the drive gears 76 which are in mesh with the worm gear teeth 71 of the drive sleeve 62. The endless track device 40 is tensioned between the drive gears 76 and the idler rollers 51-53. Positions of the drive gears 76 are overlapped with the idler rollers 51-53 in the radial direction of the support sleeve 42. The endless track device 40 is curved in a W shape between the drive gears 76 and the idler rollers 51-53.

A distal opening 63c is formed at a distal end of the barrel sleeve 63, and receives a tip of the shaft sleeve 61.

The distal cover flange 67 includes an annular ridge 67a and a cup wall 67b. The annular ridge 67a is fitted in the distal opening 63c. The cup wall 67b prevents tissue of the gastrointestinal tract from incidentally entering the propulsion assembly 20. The cup wall 67b is in a cup shape of which a diameter increases according to its distance from the annular ridge 67a. A shape of a section of the cup wall 67b is triangular and similar to an inner surface of the support sleeve 42, and is smaller than the support sleeve 42.

The end ring 66 is formed triangularly. A ring opening 66a is formed in the end ring 66 and communicates with a lumen 61a of the shaft sleeve 61. Holder recesses 66b are formed in the end ring 66 and contain respectively the first and second intermediate gears 74a and 74b in a rotatable manner. The first and second intermediate gears 74a and 74b are meshed with the spur gear teeth 72 of the drive sleeve 62. Two holes (not shown) are formed through the end ring 66, and cause the wire devices 25a and 25b to pass for connection to the first and second intermediate gears 74a and 74b.

The proximal cover flange 68 is structurally the same as the distal cover flange 67, and includes an annular ridge 68a and a cup wall 68b. The annular ridge 68a is fitted in the ring opening 66a of the end ring 66.

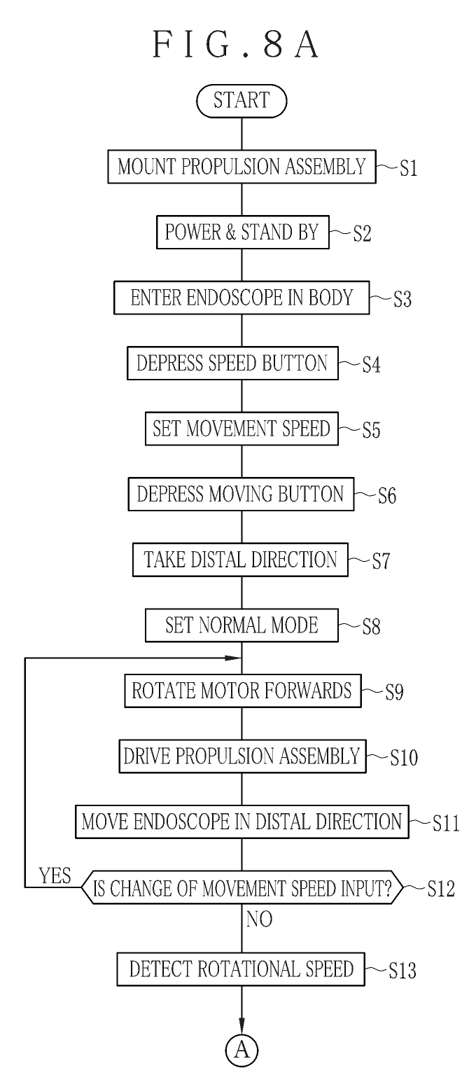

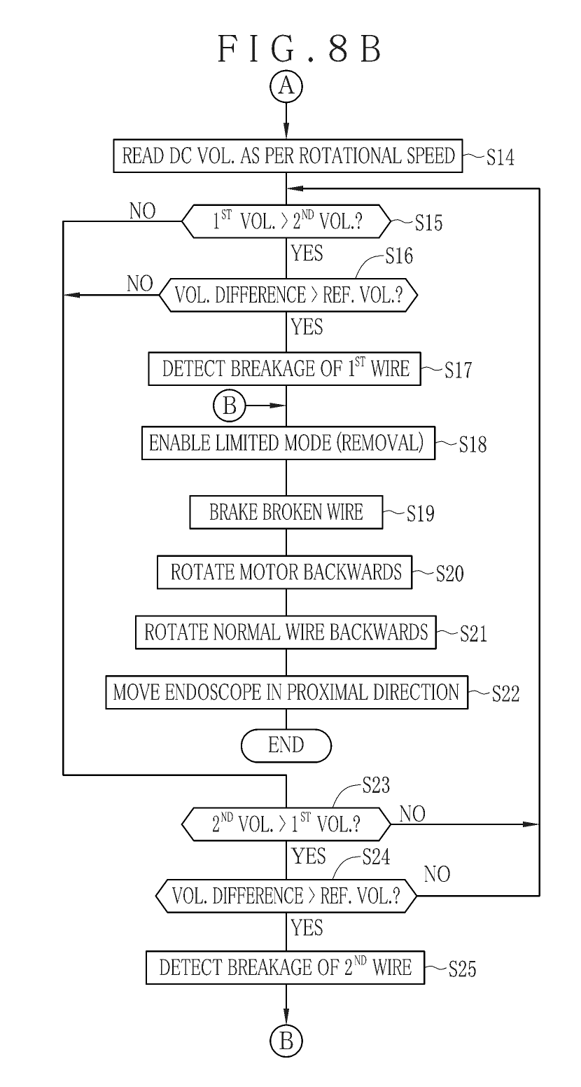

The operation of the guide apparatus 30 is described now. In FIGS. 8A and 8B, the head assembly 3a of the endoscope 2 is entered in the lumen 61a of the shaft sleeve 61 for mounting the propulsion assembly 20 on the head assembly 3a in the step S1. Then the power source for the processing apparatus, the light source apparatus and the button panel 27 is turned on for standby in the step S2. The head assembly 3a of the endoscope 2 is entered in the gastrointestinal tract of the patient in the step S3 when the examination is ready.

When the head assembly 3a advances to a predetermined site in the gastrointestinal tract, for example, short of a sigmoid colon, the speed button 29 of the button panel 27 is depressed in the step S4, to determine a movement speed of the propulsion assembly 20 in the step S5. The moving button 28 is depressed in the step S6, to input a signal for advance in the step S7. The mode setting unit 36 checks the normal mode in the step S8. The motor driver 37 drives the motor 21 to rotate forwards at the input rotational speed in the step S9. Note that the movement speed of the propulsion assembly 20 can be determined before entry of the head assembly 3a in the gastrointestinal tract.

The motor 21 rotates the wire devices 25a and 25b in a predetermined direction by use of the motor gear 22, the differential device 23 and the transmission gear train 24. The first and second intermediate gears 74a and 74b are rotated by the wire devices 25a and 25b. The spur gear teeth 72 are rotated by the first and second intermediate gears 74a and 74b, so as to rotate the drive sleeve 62.

As the drive sleeve 62 is rotated, the drive gears 76 rotate in mesh with the worm gear teeth 71. In response, the lower run 48 of the endless track device 40 is moved in the step S10 in the propulsion direction in FIG. 5 between the drive gears 76 and the idler rollers 51-53. The outer surface 40b of the upper run 46 of the endless track device 40 moves in the proximal direction outside the support sleeve 42 in contact with the body tissue of the gastrointestinal tract. The outer surface 40b of the lower run 48 inside the support sleeve 42 moves in the distal direction for turning around the endless track device 40 endlessly.

The upper run 46 of the endless track device 40 contacts body tissue of the gastrointestinal tract. When the endless track device 40 turns around, the endless track device 40 exerts force of propulsion in the proximal direction which is opposite to the distal direction of movement of the head assembly 3a. The propulsion assembly 20 propels the head assembly 3a of the endoscope 2 by exerting force to the body tissue from the distal side in the proximal direction in the step S11. Also, the propulsion assembly 20 returns the head assembly 3a by exerting force to the body tissue from the proximal side in the distal direction.

When the speed button 29 of the button panel 27 is depressed to input a control signal (yes in the step S12), the motor driver 37 changes the speed of the motor 21 to change the movement speed of the propulsion assembly 20. When the moving button 28 of the button panel 27 is depressed to input a control signal for return, the motor 21 is rotated backwards to move the propulsion assembly 20 in the proximal direction. When the moving button 28 is depressed to input a control signal for stop, the motor 21 is stopped to stop the propulsion assembly 20. Consequently, it is possible to propel the head assembly 3a of the endoscope 2 to a desired site in the gastrointestinal tract by combination of those functions.

Light from the light source apparatus is guided through the light guide device 18 and the lighting windows 8a and 8b, and applied to an object in a body cavity. The CCD in the head assembly 3a detects object light from the object, and outputs an image signal. The image signal is input to a processing apparatus through the signal line 17 and the universal cable 5, for a display panel (not shown) to display an image. A doctor or operator observes the inside of the body cavity with the display panel.

When he or she discovers a lesion in the object of interest by imaging, he or she protrudes a suitable type of a medical instrument from the distal instrument opening 9 by passage through the proximal instrument opening 13 for treatment of the lesion.

To clean up the imaging window 7, the fluid supply buttons 15 are operated to supply fluid through the fluid supply channel 16 to the end nozzle 10, for example air or water from the fluid supply apparatus. The air or water is ejected by the end nozzle 10 to the imaging window 7 to remove dust. After the imaging, the motor 21 rotates backwards. The propulsion assembly 20 turns around for return, to remove the endoscope 2 from the body cavity by proximal movement.

In the step S13, the first tachogenerator 33 detects a rotational speed of the first wire device 25a while the propulsion assembly 20 moves endlessly. The second tachogenerator 34 detects a rotational speed of the second wire device 25b. The tachogenerators 33 and 34 generate direct current voltages (DC voltages) according to the rotational speeds, and send the voltages to the controller 26 in the step S14. The failure detector 38 of the controller 26 for fail-safe control detects wire breakage of the wire devices 25a and 25b according to the direct current voltages from the tachogenerators 33 and 34. Such breakage is very likely to occur between the actuating mechanism 35 and the propulsion assembly 20.

If the first wire device 25a is broken, load to the first wire device 25a decreases, and load to the second wire device 25b increases. In the differential device 23, the pinion gear, which is disposed between the first and second wire gears for the wire devices 25a and 25b, is rotated. The first wire gear is caused to rotate at an additional amount according to a rotation amount of the pinion gear, to increase a rotational speed of the first wire device 25a. The failure detector 38 operates on the basis of the function of the differential device 23. If a first voltage of the first tachogenerator 33 is higher than a second voltage of the second tachogenerator 34 (yes in the step S15), and if a difference between the first and second voltages (voltage difference) is higher than the reference voltage (yes in the step S16), then the failure detector 38 detects breakage of the first wire device 25a according to the first voltage in the step S17.

When the failure detector 38 detects breakage of the first wire device 25a, the mode setting unit 36 disables the normal mode and sets a removal mode as a limited mode in the step S18 for fail-safe control in which the propulsion assembly 20 is enabled only to move in the proximal direction. In the removal mode, the mode setting unit 36 drives the first brake device 31 to brake the first wire device 25a in the step S19. Then the mode setting unit 36 causes the motor driver 37 to rotate the motor 21 in the backward direction in the step S20. The first wire device 25a is braked while the motor 21 rotates backwards, and can be prevented from rotating idly. The rotation of the motor 21 is transmitted to the second wire device 25b being normal, so that the endless track device 40 turns around endlessly in the direction for return. At the same time, the pinion gear rotates as the first wire device 25a is stopped. An increase in the rotational speed of the second wire device 25b occurs according to the rotation amount of the pinion gear.

In the removal mode, the controller 26 rotates the motor 21 in the backward direction to rotate the second wire device 25b backwards in the step S21. The propulsion assembly 20 is moved in the proximal direction to return the head assembly 3a of the endoscope 2 in the step S22. It is possible to remove the propulsion assembly 20 from out of the body cavity even if the first wire device 25a is broken down. In the removal mode, the movement speed can be changed by use of the speed button 29. However, the distal direction of moving the propulsion assembly 20 cannot be selected, because the button operation for setting the distal direction of movement is invalidated.

If the second voltage is higher than the first voltage (no in the step S15 or S16 and yes in the step S23), and if the voltage difference is higher than the reference voltage (yes in the step S24), then the failure detector 38 detects breakage of the second wire device 25b according to the second voltage in the step S25.

As the breakage of the second wire device 25b is detected by the failure detector 38, the mode setting unit 36 disables the normal mode and sets the removal mode, and drives the second brake device 32 to brake the second wire device 25b in the step S19. In the removal mode, the motor 21 is rotated in the backward direction to rotate the first wire device 25a backwards in the steps S20 and S21. The head assembly 3a of the endoscope 2 is moved in the proximal direction in the step S22.

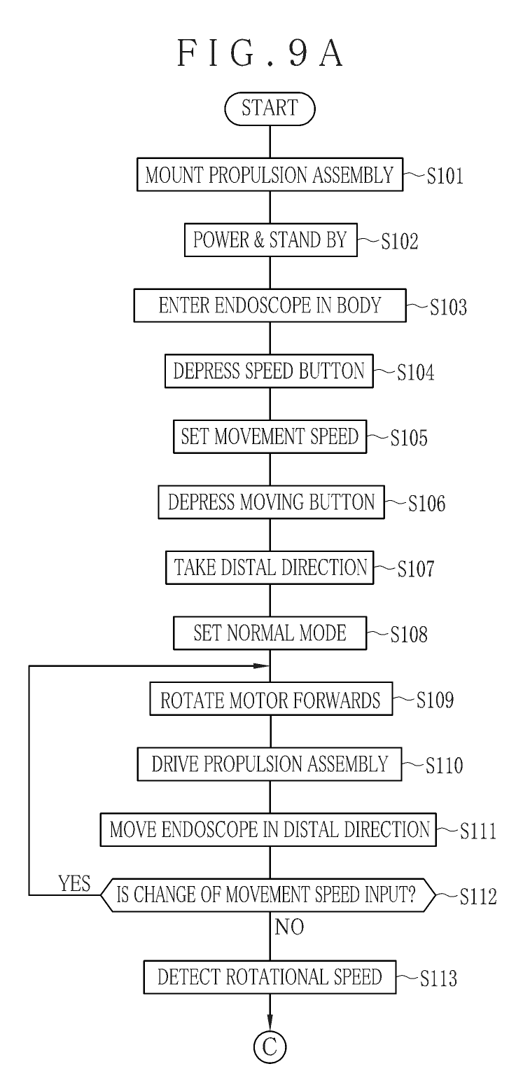

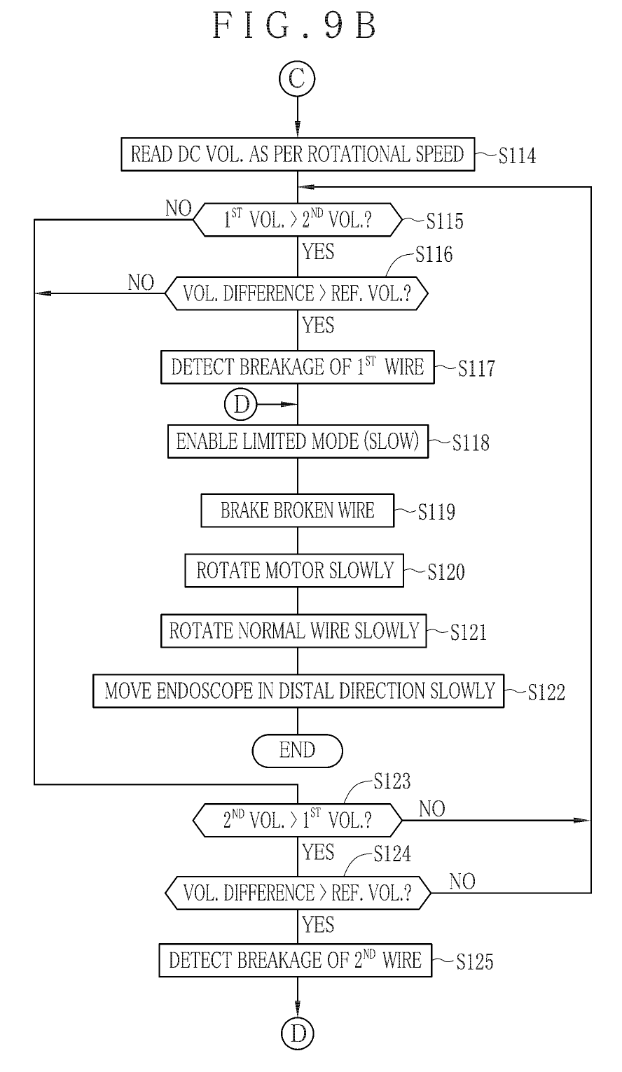

In FIGS. 9A and 9B, another preferred fail-safe control is illustrated, in which a normal wire device is rotated slowly upon detecting breakage. Elements similar to those of the above embodiment are designated with identical reference numerals.

In FIGS. 9A and 9B, the propulsion assembly 20 is mounted on the head assembly 3a of the endoscope 2 in the step S101. The power source is turned on for standby in the step S102. The head assembly 3a is entered in a gastrointestinal tract of a patient in the step S103 when the examination is ready. Steps S104-S116 and S123-S125 are the same as the steps S4-S16 and S23-S25 of the above embodiment, and are not described further.

When the failure detector 38 detects breakage of the first wire device 25a in the step S117, the controller 26 disables the normal mode and sets a slow mode as a limited mode for fail-safe control in the step S118. The first wire device 25a is braked in the step S119. The motor 21 is caused to rotate at a low speed in the step S120, to rotate the second wire device 25b slowly in the step S121. Thus, the propulsion assembly 20 moves at a low speed in the distal direction together with the head assembly 3a of the endoscope 2 in the step S122. Consequently, it is possible to reduce the risk of breakage of the second wire device 25b in comparison with rotation at a high speed. In the slow mode, directions of moving the propulsion assembly 20 are selectable so that the propulsion assembly 20 can return. The movement speed cannot be changed, because the operation of the speed button 29 for setting the speed is invalidated.

If the second voltage is higher than the first voltage (no in the step S115 or S116 and yes in the step S123), and if the voltage difference is higher than the reference voltage (yes in the step S124), then the failure detector 38 detects breakage of the second wire device 25b according to the second voltage in the step S125.

As the failure detector 38 detects breakage of the second wire device 25b, the controller 26 disables the normal mode and sets the slow mode in the step S118, and drives the second brake device 32 to brake the second wire device 25b in the step S119. The motor driver 37 rotates the motor 21 at a low speed to rotate the first wire device 25a slowly in the steps S120 and S121. The propulsion assembly 20 is moved in the distal direction at a low speed by means of the first wire device 25a. The head assembly 3a of the endoscope 2 moves together at the low speed in the step S122.

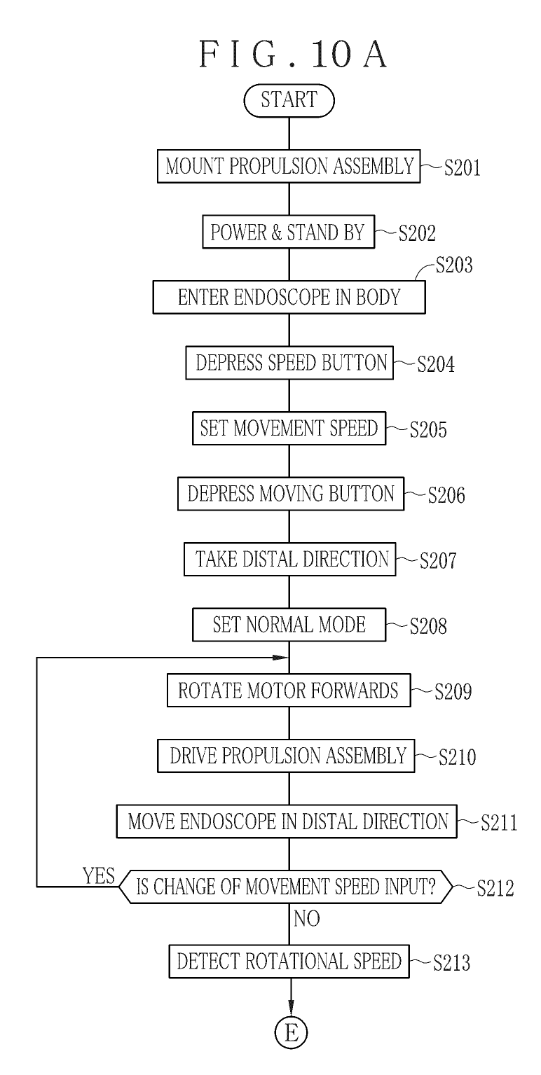

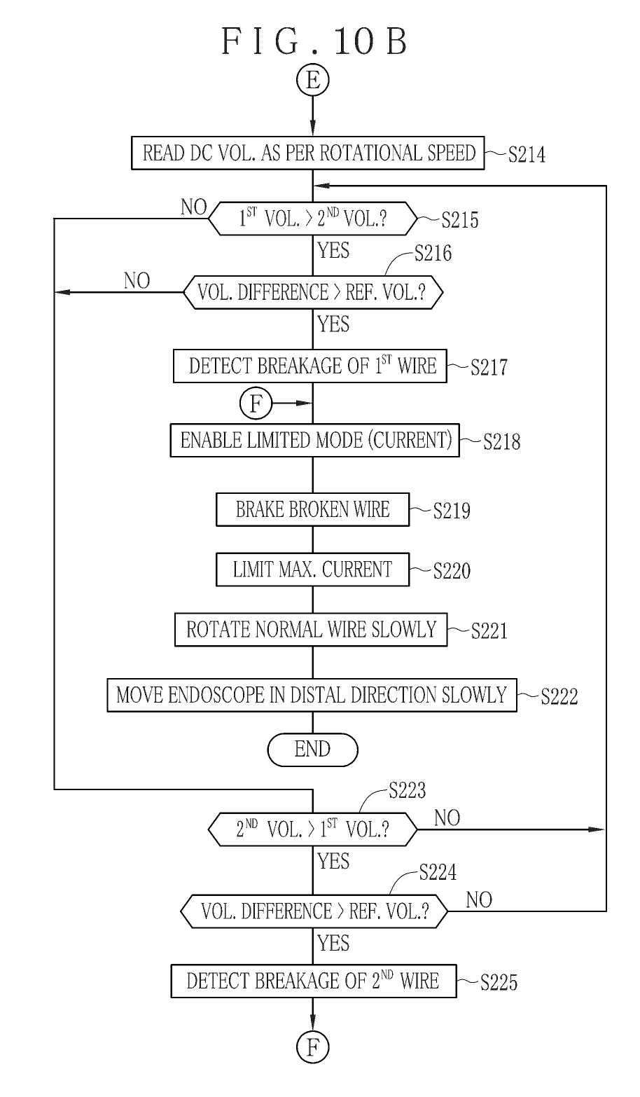

In FIGS. 10A and 10B, still another preferred fail-safe control is illustrated, in which a current limiting function is used for the motor 21 upon breakage of a wire. Elements similar to those of the above embodiments are designated with identical reference numerals.

In FIGS. 10A and 10B, the propulsion assembly 20 is mounted on the head assembly 3a of the endoscope 2 in the step S201. The power source is turned on for standby in the step S202. The head assembly 3a is entered in a gastrointestinal tract in the step S203 when the examination is ready. Steps S204-S216 and S223-S225 are the same as the steps S4-S16 and S23-S25 of the above embodiment.

When the failure detector 38 detects breakage of the first wire device 25a in the step S217, the controller 26 disables the normal mode and sets a low current mode (current limiting) as a limited mode for fail-safe control in the step S218. The first wire device 25a is braked in the step S219. Also, the mode setting unit 36 sets the maximum current of the motor in the step S220. This is effective in disabling manual operation for increasing the rotational speed over the limit speed for safety even if a user operates the button panel 27 for quickly rotating the motor 21. The motor 21 is caused to rotate at a low speed to rotate the second wire device 25b slowly in the step S221. Thus, the propulsion assembly 20 moves at a low speed in the direction for propulsion together with the head assembly 3a of the endoscope 2 in the step S222. Consequently, it is possible to reduce the risk of breakage of the second wire device 25b in comparison with rotation at a high speed. In the low current mode, directions of moving the propulsion assembly 20 can be selected. However, the movement speed is limited, because the operation of the speed button 29 for setting the speed is invalidated.

Furthermore, the three embodiments of the limited mode described above can be combined with one another for fail-safe control. For example, a normal wire device can be driven slowly in a backward direction when a particular wire device is broken. Also, a normal wire device can be driven with a limited current in a backward direction when a particular wire device is broken.

In the first embodiment, a normal wire device is rotated in a backward direction at a high speed when one wire device is broken. However, the normal wire device may be rotated backwards at any of a high speed, low speed and normal speed. Its rotational speed can be variable according to manual operation of the speed button 29.

In the above embodiments, rotational speeds of the first and second wire devices are detected. If the rotational speeds are higher than the reference speed, it is judged that one of the wire devices is broken down. However, a method of failure detection of the wire devices can be modified suitably for the purpose.

In the above embodiments, a profile surface of the support sleeve 42 is circular as viewed in a cross section. However, the support sleeve 42 can be formed in a polygonal shape, for example, triangular or quadrangular shape as viewed in a cross section.

Also, the inner surface of the support sleeve 42 and a profile surface of the barrel sleeve 63 are triangular or polygonal as viewed in a cross section in the above embodiments, but can be formed cylindrically.

In the above embodiments, the endless track device is in a toroidal shape. However, an endless track device of the invention may include a plurality of endless belts arranged in a circumferential direction of the support sleeve and extending in the axial direction.

In the above embodiments, the drive gears 76 are rotatable between the worm gear teeth 71 and the endless track device 40. However, it is possible structurally to cause the worm gear teeth 71 to drive the endless track device 40 directly without use of the drive gears 76. To this end, circumferential directions of the motor and wire device should be determined suitably, because a circumferential direction of the worm gear teeth 71 for propulsion is opposite between structures with and without the drive gears 76.

In the first embodiment, the differential device 23 is used mechanically for rotating the normal wire device at a high speed. However, the normal wire device may be rotated at the high speed by electric control of a current to the motor 21. In the second and third embodiments, the motor 21 is electrically controlled for rotating the normal wire device at a low speed. However, the normal wire device can be rotated at the low speed by mechanical control, for example, a speed reducing gear set.

In any of the above embodiments, it is possible not to use the differential device 23. Instead, a simple transmission mechanism can operate for transmitting rotation of the motor 21 to the two wire devices.

In the above embodiments, the endoscope is for a medical use. However, an endoscope of the invention can be one for industrial use, a probe of an endoscope, or the like for various purposes.

Although the present invention has been fully described by way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. A guide apparatus for an endoscope having a section of an elongated tube for entry in a body cavity, comprising:
a propulsion assembly, having an endless track device, for mounting on a head assembly of said elongated tube, said endless track device moving endlessly in said body cavity to propel said head assembly;
a motor disposed discretely from said endoscope;
plural wire devices for transmitting rotation of said motor to move said endless track device;
a differential device, disposed between said wire devices and said motor, for changing a rotational speed of said wire devices according to load applied to said wire devices;
plural brake devices for braking respectively said wire devices;
a failure detector for, if one of said wire devices is broken, detecting said broken wire device according to a change in said rotational speed;
a controller for actuating one of said brake devices corresponding to said broken wire device, to transmit rotation of said motor to a normal one of said wire devices.

2. A guide apparatus as defined in claim 1, wherein said differential device is a differential gear device.

3. A guide apparatus as defined in claim 2, wherein when said failure detector detects said broken wire device, said controller disables a normal mode for said motor without limitation, and sets a limited mode for limited operation of said motor relative to said normal mode.

4. A guide apparatus as defined in claim 3, wherein in said limited mode, said motor is rotated backwards to move said elongated tube in a proximal direction.

5. A guide apparatus as defined in claim 3, wherein in said limited mode, said motor is rotated at a low speed.

6. A guide apparatus as defined in claim 3, wherein said failure detector includes:
plural speed sensors for detecting said rotational speed of respectively said wire devices;
an arithmetic unit for comparing said rotational speed from said speed sensors, and determining that one of said wire devices of which said rotational speed is higher is said broken wire device if a difference in said rotational speed becomes equal to or more than a reference value.

7. A guide apparatus as defined in claim 3, wherein said propulsion assembly includes:
a shaft sleeve for mounting on said head assembly of said elongated tube;
a support sleeve, disposed around said shaft sleeve, for supporting said endless track device movably along inner and outer surfaces thereof;
a driving device for driving said endless track device.

8. A guide apparatus as defined in claim 7, wherein said driving device includes:
a drive sleeve, surrounded in said support sleeve, supported around said shaft sleeve in a rotatable manner, and rotated by said wire devices;
worm gear teeth formed around said drive sleeve;
a barrel sleeve disposed between said drive sleeve and said support sleeve;
plural drive gears, arranged on said support sleeve in a circumferential direction, rotated by said worm gear teeth, for endlessly moving said endless track device;
plural idler rollers, arranged on said support sleeve, for tensioning said endless track device in cooperation with said drive gears.

9. A guide apparatus as defined in claim 8, wherein said barrel sleeve has three walls arranged triangularly as viewed in a cross section, and each one of said drive gears is disposed on one of said walls;
said inner surface of said support sleeve is shaped triangularly to correspond to said barrel sleeve, and said outer surface is cylindrical.

10. A guide apparatus as defined in claim 9, wherein said endless track device is a device having an annular surface and extending to cover said support sleeve, or includes plural endless belts.
